(19) United States
(12) Patent Application Publication
(10) Pub. No.: US 2010/0008960 A1
(43) Pub. Date: Jan. 14, 2010

(54) Beauty/medication patch

(76) Inventors: Hsien Kai MENG, Taipei City (TW); Shu Pin HSIEH, Taipei City (TW); Yuan Yi Yeh, Sanchong City (TW); Chang Wei Lin, Kaohslung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/585,466

(22) Filed: Sep. 16, 2009

Related U.S. Application Data
(62) Division of application No. 11/979,388, filed on Nov. 2, 2007.

(30) Foreign Application Priority Data
Apr. 25, 2007 (TW) 096114662

(51) Int. Cl. A61K 8/02; A61K 47/34; A61K 47/36; A61K 47/42; A61Q 19/00

(52) U.S. Cl. 424/401; 514/772.3; 514/774; 514/777; 514/773

(57) ABSTRACT

A beauty/medication patch including a substrate, an outer layer, and a medication lotion; the outer layer being made of a degradable material being disposed on the substrate; one or a plurality of micro-probe array being disposed on a surface of the outer layer; the medication lotion being coated between the substrate and the outer layer, and/or admixed in the substrate; the patch being attached to a human skin, the micro-probe array piercing through a stratum corneum layer of the human skin and naturally being degraded by human skin or absorbed and metabolized by a human body; and the medication lotion penetrating into the skin and being absorbed by the human skin.

### RELATED APPLICATIONS

This application is a Divisional patent application of co-pending application Ser. No. 11/979,388, filed on 2 Nov. 2007. The entire disclosure of the prior application, Ser. No. 11/979,388, from which an oath or declaration is supplied, is considered a part of the disclosure of the accompanying Divisional application and is hereby incorporated by reference.

### BACKGROUND OF THE INVENTION

1. Field of the Invention

The present invention is related to a beauty/medication patch, and more particularly, to one made of a degradable outer layer and a micro probe array to help absorption of medication lotion by human skin tissue.

2. Description of the Prior Art

A human skin structure includes four layers, respectively stratum corneum layer, epidermis, dermis, and hypodermic layer. As the outer skin, the stratum corneum layer is composed of dead cells to form a diffusion barrier to further obstruct any skin care product applied thereon. The absorption of the skin therefore is restricted since the medical media is prevented from penetrating into the epidermis or even the dermis. Besides, it is not easy for cells of the outer skin to undergo the normal come-off and will cause the skin to get roughen, lose its gloss, reject cosmetics, and further affect its metabolism.

Face masks for beauty purpose generally available in the market have coated or admixed a layer of extract, medication lotion or other equivalent material on its inner side. When applied on a face of a user for a while, the stratum corneum layer of the skin absorbs the extract or lotion on the patch resulting in a glossy and moisturized face. However, the results are short-lived due to that the extract or lotion from the patch fails to fully enter into the epidermis or the derma; that is, the amount of the extract or lotion having penetrated into the epidermis or even further into the derma through the stratum corneum layer is very limited; and the expected results from the extract or lotion is significantly affected. As the primary diffusion barrier, the stratum corneum layer claims 70% up to 90% of obstruction characteristics of the skin.

In comparison with the stratum corneum layer of the skin, the epidermis comprised of live tissues has higher water content and gives better permeability to skin care products. The area near where the derma and epidermis contact each other is elated to a tissue containing rich capillary follicles. Should the skin care product be able to reach the epidermis, ingredients of the skin care product are able to fast spread to the dermis. At present, face masks available in the market could at their best provide temporary supplement of water content to the facial skin. No improvements have been made yet in the structure of the face mask to overcome the diffusion barrier of the stratum corneum layer for extract or lotion of the skin care product to penetrate into the epidermis.

### SUMMARY OF THE INVENTION

The primary purpose of the present invention is to provide a beauty/medication patch that is capable of penetrating into human skin and having medication lotion coated on the patch to be well absorbed by human skin. To achieve the purpose, the present invention includes a substrate, an outer layer, and or at least one medication lotion. Wherein, the outer layer is made of degradable material; one or a plurality of micro-probe array is formed on a surface of the outer layer; and the medication lotion is coated between the substrate and the outer layer and/or admixed in the substrate. Once the beauty/medication patch is attached to human skin, the micro-probe array pierces into the stratum corneum layer of the human skin to be naturally degraded or absorbed and metabolized by human skin so to allow the medication lotion to penetrate into and get absorbed by human skin.

Another purpose of the present invention is to provide a beauty/medication patch wherein the medication lotion is admixed in the outer layer. Once the beauty/medication patch is attached to human skin, the micro-probe array pierces into the stratum corneum layer of the human skin to be naturally degraded or absorbed and metabolized by human skin so as to allow the medication lotion to penetrate into and get absorbed by human skin.

Another purpose yet of the present invention is to provide a beauty/medication patch wherein the medication lotion is coated on the micro-probe array on the outer layer. Once the beauty/medication patch is attached to human skin, the micro-probe array pierces into the stratum corneum layer of the human skin to be naturally degraded or absorbed and metabolized by the human skin so as to allow the medication lotion to penetrate into and get absorbed by human skin.

### BRIEF DESCRIPTION OF THE DRAWINGS

FIG. 1 is a sectional view showing a first preferred embodiment of a beauty/medication patch of the present invention.

FIG. 2 is another sectional view showing the first preferred embodiment of a beauty/medication patch of the present invention.

FIG. 3 is a perspective view showing the first preferred embodiment of a beauty/medication patch of the present invention.

FIG. 4 is a sectional view showing a second preferred embodiment of a beauty/medication patch of the present invention.

FIG. 5 is a sectional view showing a third preferred embodiment of a beauty/medication patch of the present invention.

### DETAILED DESCRIPTION OF THE PREFERRED EMBODIMENTS

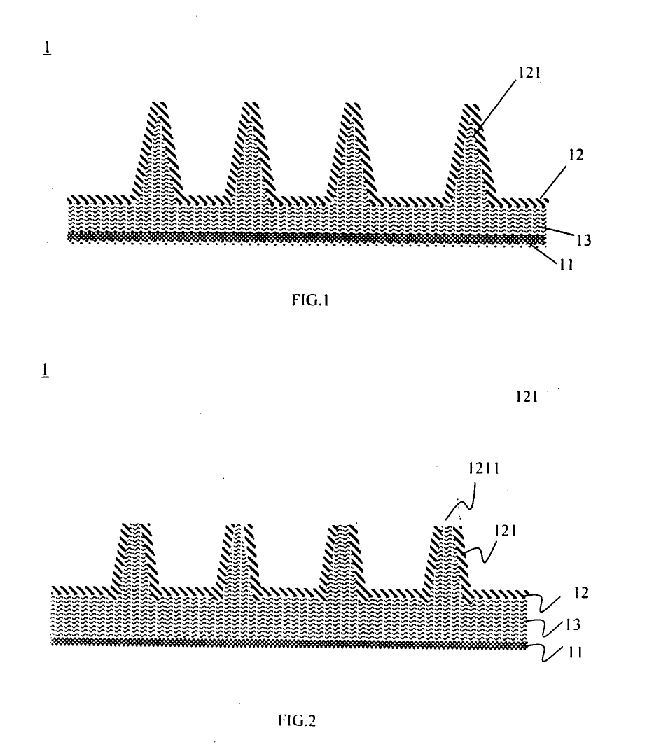

Referring to FIG. 1 showing a sectional view of a beauty/medication patch according to a preferred embodiment of the present invention, a beauty/medication patch 1 includes a substrate 11, an outer layer 12, and a medication lotion 13. The outer layer 12 made of at least one degradable material is disposed on the substrate and one or a plurality micro-probe array 121 is formed on a surface of the outer layer 12. The medication lotion 13 is coated between the substrate 11 and the outer layer, and/or admixed in the substrate 11. When the beauty/medication patch 1 is attached to a human skin, the micro-probe array 121 pierces through the stratum corneum layer of the human skin and subsequently as illustrated in FIG. 2 for the cross-sectional drawing of a beauty/medication patch according to a preferred embodiment of the present invention, the micro-probe array 121 piercing through the stratum corneum layer of the human skin will be naturally degraded by human skin or absorbed and metabolized by a human body so to permit the medication lotion to penetrate into the skin and to be absorbed anatomically by the human skin.

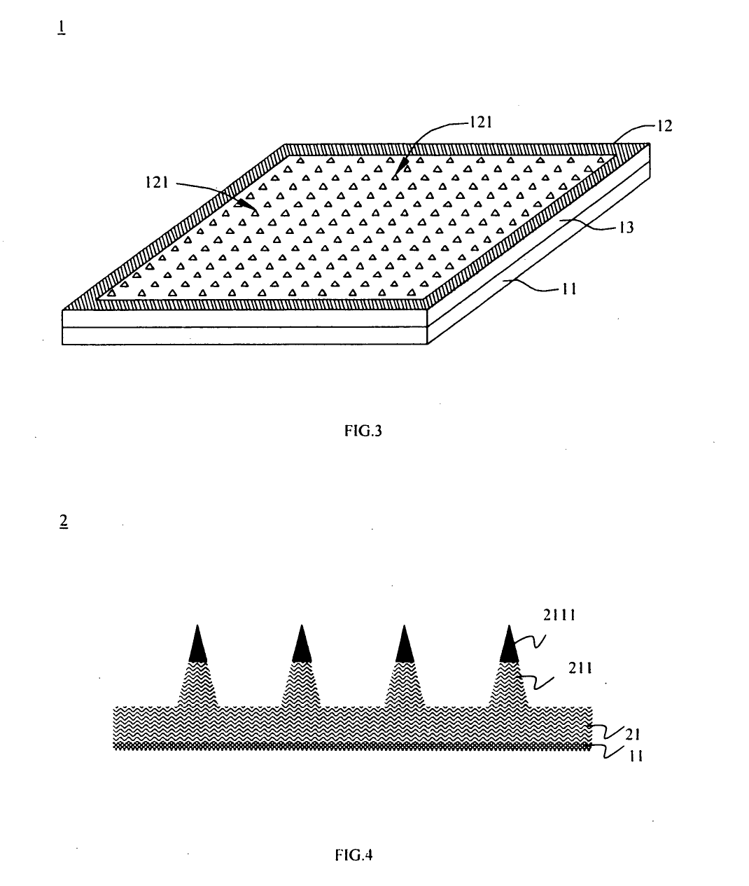

Now referring to FIG. 3 for a perspective view of the first preferred embodiment wherein a beauty/medication patch 1 includes a substrate 11, an outer layer 12, and a medication lotion 13. The outer layer 12 made of a degradable material is disposed on the substrate and at least one micro-probe array 121 is formed on a surface of the outer layer 12. The medication lotion 13 is coated between the substrate 11 and the outer layer, and/or admixed in the substrate 11. When the beauty/medication patch 1 is attached to a human skin, the micro-probe array 121 pierces through the stratum corneum layer of the human skin and subsequently as illustrated in FIG. 2, the micro-probe array 121 piercing through the horny layer of the human skin will be naturally degraded by human skin or absorbed and metabolized by human body so as to permit the medication lotion to penetrate into the skin and to be absorbed anatomically by the human skin.

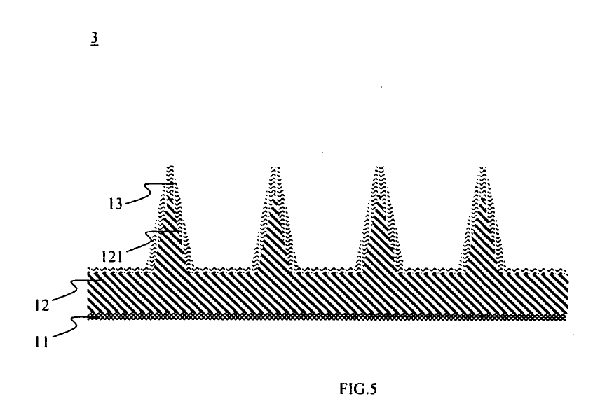

As illustrated in FIG. 4 for the cross-sectional drawing of a beauty/medication patch according to a second preferred embodiment of the present invention, a patch 2 includes a substrate 11 and an outer layer 21. The outer layer 21 is mixed with a degradable material and a medication lotion on the substrate. A surface of the outer layer 21 is formed with at least one micro-probe array 211. When the beauty/medication patch 2 is attached to a human skin, the micro-probe array 211 pierces through a stratum corneum layer of the human skin, and a partial area 2111 of the micro-probe array 211 piercing through the stratum corneum layer of the human skin will be naturally degraded by human skin or absorbed and metabolized by human body so as to permit the medication lotion mixed in the degraded area 2111 to penetrate into the skin and to be absorbed anatomically by the human skin.

In a third preferred embodiment of the present invention as illustrated in FIG. 5, a beauty/medication patch 3 includes a substrate 11, an outer layer 12, and a medication lotion 13. The outer layer 12 comprised of at least one degradable material is disposed on the substrate 12, and at least one micro-probe array 121 is formed on a surface of the outer layer. The medication lotion 13 is coated on the micro-probe array 121 on top of the outer layer 12. When the micro-probe array 121 of the beauty/ medication patch 3 is attached to a human skin, the micro-probe array 121 pierces through a stratum corneum layer of the human skin, and an area pierced by the micro-probe array 121 will be naturally degraded by human skin or absorbed and metabolized by the human body so as to permit the medication lotion 13 to penetrate into the skin and to be absorbed anatomically by the human skin.

The substrate 11 is made of elastic non-woven fabric, liquid gel patch, bio-fiber, or any combination thereof The outer layers 12, 21 are made of a degradable material including Aliphatic Polyester, Poly Cyano Acrylate, Poly Amides, Poly Acetals, Collagen, Gelatin, Hyaluronic Acid, Chitin, Chitosan, POE, PAH, PLA, PGA, PLGA, or any combination thereof. The micro-probes 121, 211 are made of a degradable material selected from Aliphatic Polyester, Poly Cyano Acrylate, Poly Amides, Poly Acetals, Collagen, Gelatin, Hyaluronic Acid, Chitin, Chitosan, POE, PAH, PGA, PLGA, or any combination thereof. The medication lotion 13 includes collagen, placenta, hyaluronic acid, L-(+) Ascorbic Acid, synthetic enzyme Q10, vitamins (e.g., Vitamin E), polyphenols (e.g., grape seed polyphenols ) or any combination thereof. The height of the mciro-probe array 121, 211 fall within a range of 20 and 600 microns.

It is to be noted that the preferred embodiments disclosed in the specification and the accompanying drawings are not limiting the present invention; and that any construction, installation, or characteristics that is same or similar to that of the present invention should fall within the scope of the purposes and claims of the present invention.

What is claimed is:

1. A beauty/medication patch comprising a substrate; an outer layer made of a degradable material and disposed on the substrate and at least one micro-probe array disposed on a surface of the outer layer; and a medication lotion coated between the substrate and the outer layer, and/or admixed in the substrate; the beauty/medication patch being attached to a human skin; the micro-probe array piercing through a stratum corneum layer of the human skin and naturally being degraded by human skin or absorbed and metabolized by a human body; and the medication lotion penetrating into the skin and being absorbed by the human skin.

2. The beauty/medication patch as claimed in claim 1, wherein the outer layer is made of a degradable material including Aliphatic Polyester, Poly Cyano Acrylate, Poly Amides, Poly Acetals, Collagen, Gelatin, Hyaluronic Acid, Chitin, Chitosan, POE, PAH, PLA, PGA, PLGA, or any combination thereof.

3. A beauty/medication patch comprising a substrate and an outer layer; the outer layer mixed with a degradable material and a medication lotion on the substrate; a surface of the outer layer being formed with at least one micro-probe array; the beauty/medication patch being attached to a human skin; the micro-probe array piercing through a stratum corneum layer of the human skin and naturally being degraded by human skin or absorbed and metabolized by a human body; and the medication lotion penetrating into the skin and being absorbed by the human skin.

4. The beauty/medication patch as claimed in claim 3, wherein the outer layer is made of a degradable material selected from Aliphatic Polyester, Poly Cyano Acrylate, Poly Amides, Poly Acetals, Collagen, Gelatin, Hyaluronic Acid, Chitin, Chitosan, POE, PAH, PGA, PLGA, or any combination thereof.

5. A beauty/medication patch includes a substrate, an outer layer, and a medication lotion; the outer layer made of a degradable material and dispose on the substrate, and at least one micro-probe array formed on a surface of the outer layer; and the medication lotion coated on the micro-probe array on top of the outer layer; the beauty/medication patch being attached to a human skin; the micro-probe array piercing through a stratum corneum layer of the human skin and naturally being degraded by human skin or absorbed and metabolized by a human body; and the medication lotion penetrating into the skin and being absorbed by the human skin.

6. The beauty/medication patch as claimed in claim 1, wherein the outer layer is made of a degradable material including Aliphatic Polyester, Poly Cyano Acrylate, Poly Amides, Poly Acetals, Collagen, Gelatin, Hyaluronic Acid, Chitin, Chitosan, POE, PAH, PLA, PGA, PLGA, or any combination thereof.
